(19) United States
(12) Patent Application Publication
(10) Pub. No.: US 2025/0031742 A1
(43) Pub. Date: Jan. 30, 2025

(54) GEL-LIKE FOOD SUPPLEMENT COMPOSITION

(71) Applicant: TECHTURE OÜ, Tallinn (EE)

(72) Inventor: Tanel SAUL, Tallinn (EE)

(73) Assignee: TECHTURE OÜ, Tallinn (EE)

(21) Appl. No.: 18/713,829

(22) PCT Filed: Dec. 8, 2021
(86) PCT No.: PCT/EP2021/084865

§ 371 Date: May 28, 2024

(51) Int. Cl. A23L 33/165; A23L 29/256; A23L 29/269; A23L 33/115

(52) U.S. Cl. CPC A23L 33/165; A23L 29/256; A23L 29/27; A23L 33/115

(57) ABSTRACT

A gel-like food supplement composition including at least one active ingredient, at least one solvent and at least one excipient, the active ingredient including at least one organic magnesium salt. The at least one organic magnesium salt is in an undissolved state, and the viscosity of the composition is 1000 to 5000 cP. The at least one organic magnesium salt includes one or more of magnesium formate, magnesium pidolate, magnesium citrate, magnesium gluconate, magnesium lactate, magnesium aspartate, magnesium ascorbate, magnesium glycinate, magnesium malate, magnesium citrate glycinate and hydrates of these salts.

### TECHNICAL FIELD

The present invention relates to a gel-like food supplement composition.

### BACKGROUND ART

Magnesium (Mg) is a chemical element that is crucial to the proper function of the body. Magnesium contributes to the maintenance of healthy bones and teeth, normal psychological function, protein synthesis and muscle function. Magnesium has an important role in the process of cell division and contributes to the reduction of tiredness and fatigue (Commission Regulation (EU) No 432/2012).

Experts say that many people are not eating enough foods rich in magnesium. Adults who consume less than the recommended amount of magnesium are more likely to have elevated inflammation markers. Inflammation, in turn, has been associated with major health conditions such as heart disease, diabetes, and certain cancers. Also, low concentrations of magnesium appear to be a risk factor for osteoporosis (https://www.webmd.com/diet/supplement-guide-magnesium#1).

Magnesium salts are essential minerals that play a critical role in the human body. It takes part in the process of energy metabolism and assists the maintenance of normal muscle function. A number of studies evaluated the correlation between magnesium status/supplementation and exercise performance, and found that the body's demand for magnesium increased as individuals' physical activity level went up. Vitamin/mineral status refers to the state of vitamin/mineral sufficiency or deficiency of any person.

Some cross-sectional surveys demonstrated a positive correlation between magnesium concentration in the body and muscle performance, including grip strength, lower-leg power, knee extension torque, ankle extension strength, maximal isometric trunk flexion, rotation, and jumping performance. Additionally, findings from intervention studies showed that magnesium supplementation might lead to improvements in functional indices such as quadriceps torque. Moreover, magnesium supplementation could improve gait speed and chair stand time in elderly women. This comprehensive review summarized the literature from both animal and human studies and aimed to evaluate scientific evidence on magnesium status/supplementation in relation to exercise performance (https://www.ncbi.nlm.nih.gov/pmc/articles/PMC5622706/).

Patent EP2253307B1 describes a high viscosity gel solution of a fully dissolved magnesium organic salt solution for feed or food applications. The gel comprises of water, preservatives and a magnesium salt which is fully dissolved and a quaternary ammonium compound-feed additive L-carnitine. A disadvantage of fully dissolving the magnesium and adding L-carnitine is that it has a higher crystallisation risk compared to the current invention. In addition, with the extremely highly concentrated solution of magnesium compound and L-carnitine product scaling and drying with repeated use is a problem. Especially with multidose pump containers. With the current invention there is no crystallisation and scaling risk and the product is easily packageable and usable in multidose containers. Furthermore, adding ammonium compounds create strong off flavours initially and stronger flavour defects during storage. L-carnitine has a sour/bitter and fishy taste and smell. In the modern market, natural and preservative free products are essential for a successful marketing of the product to the health aware consumer.

The composition described in patent EP2253307B1 is not preservative free as a significant amount of potassium sorbate (a synthetic preservative) is needed for safe storage. The current invention creates a product where preservatives are not needed for safe and long time storage.

Patent application WO2008116226A2 describes a magnesium formulation with different magnesium compounds and milk fat and lactose. The composition described is with neutral pH level and no thickener use is described. Neutral pH level products need preservatives for product microbiological safety. Current composition is acidic, thickened and packaged in airless multidose containers so there is no microbial risk.

### DISCLOSURE OF INVENTION

The invention relates to a gel-like food supplement composition with high magnesium concentration and having high viscosity in a multidose container. Said gel-like food supplement is in the form of gel or paste. Bioavailable organic magnesium salts are used in the product to achieve maximum benefits for athletes and others who need magnesium supplementation.

The gel-like food supplement composition of the invention is in the form of a high viscosity suspension (gel) or paste. The invention differs from the usual conventional single dose solid powder sachets and dilute solutions/gels available on the market at the moment (e.g. Infisport Gel Oral and Magnesium-Diasporal®) due to the facts that it is preservative free gel with high bioavailability magnesium salts and the product is extremely easy to use for the customer.

The composition of the invention comprises of one to several active ingredients, one to several excipients and one to several solvents.

Active ingredients: organic magnesium salts/compounds with any possible stoichiometry or crystal water complexes for example: formate, pidolate, citrate, gluconate, lactate, aspartate, ascorbate, glycinate, malate.

Solvents: one to several in the following selection:
Propylene glycol
Polyethylene glycol
Glycerol
Oil, plant origin (vegetable oil.)
Water

Excipients used in the composition can be as follows:
Xanthan gum as a thickener
Alginates as thickeners
Hydrogenated vegetable oil as a thickener
Natural waxes as thickeners
Sodium bicarbonate as an acidity regulator
Citric acid as an acidity regulator

The composition can be fortified with vitamins or other minerals and bioactive components to provide additional benefits. Flavouring agents can be added to obtain a desired taste. Vitamins and other bioactive components with synergistic and supportive nature especially vitamin B6, Thiamine may be added.

According to invention there is provided a gel-like food supplement composition comprising at least one active ingredient, at least one solvent and at least one excipient, said active ingredient comprises of at least one organic magnesium salt, where said organic magnesium salt is in an undissolved state, and where the viscosity of the composition is 1000 to 5000 cP.

According to invention one or several magnesium salts are selected from the group comprising magnesium formate, magnesium pidolate, magnesium citrate, magnesium gluconate, magnesium lactate, magnesium aspartate, magnesium ascorbate, magnesium glycinate, magnesium malate, magnesium citrate glycinate, calcium magnesium citrate and hydrates of these salts.

According to a first aspect of the invention, one of the excipients is a thickener.

According to a second aspect of the invention, the thickener is selected from a group comprising xanthan gum and alginates.

According to a third aspect of the invention, the cosolvent is water and the solvent is selected from the group comprising propylene glycol, polyethylene glycol and glycerol.

According to the invention, the solvent is vegetable oil.

According to the invention, preferably the thickener is selected from a group comprising hydrogenated vegetable oil and natural waxes.

According to the invention, preferably an organic magnesium salt is present in a 30-70 wt % concentration and thickener 0.1-10 wt %.

According to the invention, preferably a magnesium salt is present in a 40-60 wt % concentration and thickener 0.2-8 wt %.

### Technical Problem

Available single dose food supplements in the form of powders are prone to coagulation in prolonged storage, also they need to be dissolved (in water for example) before consumption.

Existing gel/paste form food supplements are prone to a product crystallization in prolonged storage. To overcome said problem, many of the prior art supplements contain synthetic preservatives.

### Technical Solution

The current invention is improved by incorporating more or different solvents besides water, so the magnesium organic salt is kept in a suspended form, eliminating the risk of product crystallization.

Product pH is kept acidic (in aqueous suspensions) and viscosity high (around 1000 to 5000 cP) so there is no need for synthetic preservatives. Product is homogenized for uniform particle size, distribution.

### Advantageous Effects

This invention provides a high potency magnesium supplement in compact packaging with extended in-use shelf-life. The invention improves on the previously described inventions in several key points, such as
High magnesium concentration is achieved without crystallisation risk.
The composition is storage stable without flavour defects.
The composition is preservative free with natural composition no artificial food additives are needed.

### BRIEF DESCRIPTION OF DRAWINGS

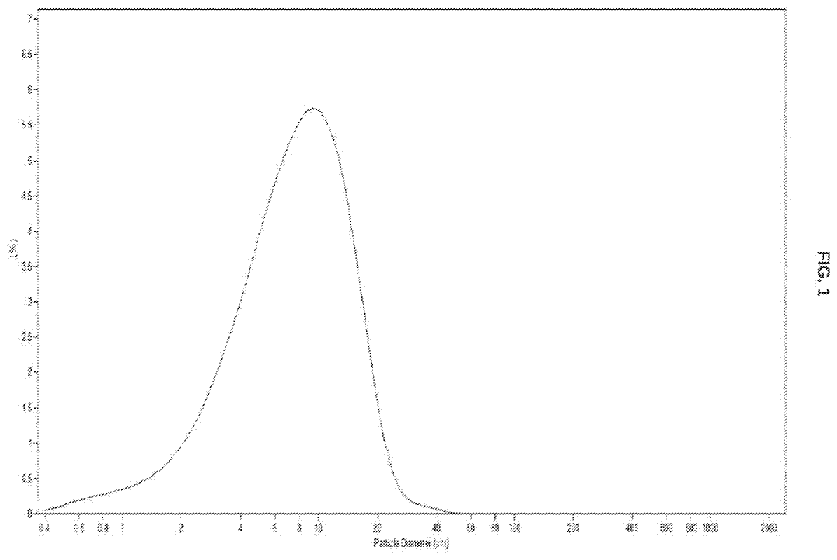

The particle size distribution of the present invention is illustrated on FIG. 1 with the optimal distance being below 20 μm. Sufficiently small particle size ensures that the product gel will not separate during storage.

### MODE(S) FOR CARRYING OUT THE INVENTION

The present invention is described with the help of exemplary embodiments. All ratios of the ingredients below are given as percentage per mass (wt %), unless specified otherwise.

### Example 1

Manufacturing process of the composition: the active ingredient (magnesium ascorbate hydrate) was dispersed in a mixture of half the total needed of solvent glycerol and 11% by weight cosolvent (water) and the mixture was homogenized with a suitable homogenizing unit at ˜3000 rpm. The thickener (sodium alginate) was dispersed in the rest of the solvent. (glycerol) separately and added to the main mixture. The mixture was homogenized again as the thickener hydrated. A sufficient amount of citric acid was added until pH 5 was achieved. Rest of the water (11% by weight) was added, and the final composition was mixed homogeneous.

TABLE 0001
Table 1

| Component | Concentration, wt % |
|---|---|
| Magnesium ascorbate hydrate | 70 |
| Glycerol | 2 |
| Sodium alginate | 5 |
| Citric acid | q.s. pH 4-5 |
| Water | 22 |

q.s. = quantity sufficient

As a result, a homogeneous paste was achieved with an acidic pH below 6 and high viscosity around 1000 to 5000 cP. Optimal particle size distribution of the composition was achieved as shown in FIG. 1. Particle size of the composition was determined with laser diffraction measurement. The gel-like suspension was packaged in airless dispensers for storage.

The product was stored for 2 years with no lumping, crystallization or flavour defect % observed. No significant microbial growth was detected within 2 years.

### Example 2

The manufacturing process of the composition described in Example 1 was used, but magnesium citrate glycinate was used as an active ingredient and xanthan gum as thickener.

TABLE 0002
Table 2

| Component | Concentration, wt % |
|---|---|
| Magnesium citrate glycinate | 50 |
| Glycerol | 25 |
| Xanthan gum | 0.30 |
| Water | 24.7 |

A homogeneous paste was achieved with an acidic pH below 6 and high viscosity around 1000 to 5000 cP. Optimal particle size distribution of the suspension was achieved according to FIG. 1. Particle size of the composition was determined with laser diffraction measurement. The suspension was packaged in airless dispensers for storage.

The product was stored for 2 years with no lumping, crystallization or flavour defects observed. No significant microbial growth was detected within 2 years.

### Example 3

The manufacturing process of the composition described in Example 1 was used, but magnesium lactate dihydrate was used as an active ingredient and polyethylene glycol as solvent.

TABLE 0003
Table 3

| Component | Concentration, wt % |
|---|---|
| Magnesium lactate dihydrate | 60 |
| Polyethylene glycol | 20 |
| Sodium alginate | 8 |
| Citric acid | q.s. pH 4-5 |
| Water | 12 |

q.s. = quantity sufficient

A homogeneous paste was achieved with an acidic pH below 6 and high viscosity around 1000 to 5000 cP. Optimal particle size distribution of the suspension was achieved according to FIG. 1. The suspension was packaged in airless dispensers for storage.

The product was stored for 2 years with no lumping, crystallization or flavour defects observed. No significant microbial growth was detected within 2 years.

### Example 4

Manufacturing process: The active ingredient (magnesium glycinate) was dispersed in approximately half the total needed weight of solvent (coconut MCT oil) and the mixture homogenized with a suitable homogenizing unit at ˜3000 rpm. The thickener (hydrogenated vegetable oil was dispersed in the rest of the solvent (coconut MCT oil) separately and added to the main mixture. The mixture was homogenized again. Solvent (coconut MCT oil) was added to make up the desired weight.

TABLE 0004
Table 4

| Component | Concentration, wt % |
|---|---|
| Magnesium glycinate | 55 |
| Coconut MCT oil | 40 |
| Hydrogenated vegetable oil | 5 |

A homogeneous paste was achieved with a high viscosity around 1000 to 5000 cP. Optimal particle size distribution of the suspension was achieved according to FIG. 1. Particle size of the composition was determined with laser diffraction measurement. The suspension was packaged in airless dispensers for storage.

The product was stored for 2 years with no lumping, crystallization or flavour defects observed. No significant microbial growth was detected within 2 years.

### Example 5

Manufacturing process: The active ingredient (magnesium citrate tribasic hydrate) was dispersed in approximately half the total needed weight of solvent (olive oil) and the mixture homogenized with a suitable homogenizing unit at ˜3000 rpm. The thickener (hydrogenated vegetable oil) was dispersed in the rest of the solvent (olive oil) separately and added to the main mixture. The mixture was homogenized again. Solvent (olive oil) was added to make up the desired weight.

TABLE 0005
Table 5

| Component | Concentration, wt % |
|---|---|
| Magnesium citrate tribasic hydrate | 60 |
| Olive oil | 35 |
| Hydrogenated vegetable oil | 5 |

A homogeneous paste was achieved with a high viscosity around 1000 to 5000 cP. Optimal particle size distribution of the suspension was achieved according to FIG. 1. Particle size of the composition was determined with laser diffraction measurement. The suspension was packaged in airless dispensers for storage.

The product was stored for 2 years with no lumping, crystallization or flavour defects observed. No significant microbial growth was detected within 2 years.

### Example 6

The manufacturing process of the composition described in Example 1 was used, but magnesium pidolate was used as an active ingredient and xanthan gum as thickener.

TABLE 0006
Table 6

| Component | Concentration, wt % |
|---|---|
| Magnesium pidolate | 45 |
| Glycerol | 28 |
| Xanthan gum | 0.50 |
| Water | 26.5 |

A homogeneous paste was achieved with an acidic pH below 6 and high viscosity around 1000 to 5000 cP. Optimal particle size distribution of the suspension was achieved according to FIG. 1. Particle size of the composition was determined with laser diffraction measurement. The suspension was packaged in airless dispensers for storage.

The product was stored for 2 years with no lumping, crystallization or flavour defects observed. No significant microbial growth was detected within 2 years.

### Example 7

Manufacturing process: The active ingredient (Magnesium formate) was dispersed in approximately half the total needed weight of solvent (coconut MCT oil) and the mixture homogenized with a suitable homogenizing unit at ˜3000 rpm. The thickener (hydrogenated vegetable oil) was dispersed in the rest of the solvent (coconut MCT oil) separately and added to the main mixture. The mixture was homogenized again. Solvent (coconut MCT oil) was added to make up the desired weight.

TABLE 0007
Table 7

| Component | Concentration, wt % |
|---|---|
| Magnesium formate | 40 |
| Coconut MCT oil | 56 |
| Hydrogenated vegetable oil | 4 |

A homogeneous paste was achieved with a high viscosity around 1000 to 5000 cP. Optimal particle size distribution of the suspension was achieved according to FIG. 1. Particle size of the composition was determined with laser diffraction measurement. The suspension was packaged in airless dispensers for storage.

The product was stored for 2 years with no lumping, crystallization or flavour defects observed. No significant microbial growth was detected within 2 years.

### Example 8

The manufacturing process of the composition described in Example 1 was used, but calcium magnesium citrate was used as an active ingredient and xanthan gum as thickener.

TABLE 0008
TABLE 8

| Component | Concentration, wt % |
|---|---|
| Calcium magnesium citrate | 55 |
| Glycerol | 23 |
| Xanthan gum | 0.30 |
| Water | 21.7 |

A homogeneous paste was achieved with an acidic pH below 6 and high viscosity around 1000 to 5000 cP. Optimal particle size distribution of the suspension was achieved according to FIG. 1. Particle size of the composition was determined with laser diffraction measurement. The suspension was packaged in airless dispensers for storage.

The product was stored for 2 years with no lumping, crystallization or flavour defects observed. No significant microbial growth was detected within 2 years.

### Example 9

The manufacturing process of the composition described in Example 1 was used, but magnesium gluconate was used as an active ingredient and xanthan gum as thickener.

TABLE 0009
Table 9

| Component | Concentration, wt % |
|---|---|
| Magnesium gluconate | 50 |
| Glycerol | 24 |
| Xanthan gum | 0.60 |
| Water | 24.4 |

A homogeneous paste was achieved with an acidic pH below 6 and high viscosity around 1000 to 5000 cP. Optimal particle size distribution of the suspension was achieved according to FIG. 1. Particle size of the composition was determined with laser diffraction measurement. The suspension was packaged in airless dispensers for storage.

The product was stored for 2 years with no lumping, crystallization or flavour defects observed. No significant microbial growth was detected within 2 years.

### Example 10

Manufacturing process: The active ingredient (Magnesium aspartate) was dispersed in approximately half the total needed weight of solvent (coconut MCT oil) and the mixture homogenized with a suitable homogenizing unit at ˜3000 rpm. The thickener (hydrogenated vegetable oil) was dispersed in the rest of the solvent (coconut MCI oil) separately and added to the main mixture. The mixture was homogenized again. Solvent (coconut MCT oil) was added to make up the desired weight.

TABLE 0010
Table 10

| Component | Concentration, wt % |
|---|---|
| Magnesium aspartate | 44 |
| Coconut MCT oil | 53 |
| Hydrogenated vegetable oil | 3 |

A homogeneous paste was achieved with a high viscosity around 1000 to 5000 cP. Optimal particle size distribution of the suspension was achieved according to FIG. 1. Particle size of the composition was determined with laser diffraction measurement. The suspension was packaged in airless dispensers for storage.

The product was stored for 2 years with no lumping, crystallization or flavour defects observed. No significant microbial growth was detected within 2 years.

### Example 11

The manufacturing process of the composition described in Example 1 was used, but magnesium malate was used as an active ingredient and xanthan gum as thickener.

TABLE 0011
Table 11

| Component | Concentration, wt % |
|---|---|
| Magnesium malate | 52 |
| Glycerol | 24 |
| Xanthan gum | 0.30 |
| Water | 23.7 |

A homogeneous paste was achieved with an acidic pH below 6 and high viscosity around 1000 to 5000 cP. Optimal particle size distribution of the suspension was achieved according to FIG. 1. Particle size of the composition was determined with laser diffraction measurement. The suspension was packaged in airless dispensers for storage.

The product was stored for 2 years with no lumping, crystallization or flavour defects observed. No significant microbial growth was detected within 2 years.

### INDUSTRIAL APPLICABILITY

The composition is relatively easily produced both at smaller and larger batch sizes. With a skilled specialist it is easily possible to scale up the batch size to a desired need, allowing for mass production.

1-8. (canceled)

9. A food supplement composition comprising at least one active ingredient, at least one solvent and at least one excipient, said active ingredient comprising at least one organic magnesium salt, wherein said at least one organic magnesium salt is in an undissolved state, wherein the viscosity of the composition is 1000 to 5000 cP, wherein the at least one organic magnesium salt is selected from the group consisting of one or more of magnesium formate, magnesium pidolate, magnesium citrate, magnesium gluconate, magnesium lactate, magnesium aspartate, magnesium ascorbate, magnesium glycinate, magnesium malate, magnesium citrate glycinate, calcium magnesium citrate and hydrates of these salts, wherein the at least one excipient comprises a thickener, wherein the at least one organic magnesium salt is present in a 30-70 wt % concentration and the thickener is present in a 0.1-10 wt % concentration, and wherein the food supplement composition is in a gel or paste form.

10. The composition according to claim 9, wherein the thickener is selected from the group consisting of xanthan gum and alginates.

11. The composition according to claim 9, wherein the solvent is selected from the group consisting of propylene glycol, polyethylene glycol and glycerol, and the composition comprises water as a cosolvent.

12. The composition according to claim 9, wherein the solvent is vegetable oil.

13. The composition according to claim 12, wherein the thickener is selected from the group consisting of hydrogenated vegetable oil and natural waxes.

14. The composition according to claim 9, wherein the at least one organic magnesium salt is present in a 40-60 wt % concentration and the thickener is present in a 0.2-8 wt % concentration.

15. The composition according to claim 10, wherein the at least one organic magnesium salt is present in a 40-60 wt % concentration and the thickener is present in a 0.2-8 wt % concentration.

16. The composition according to claim 11, wherein the at least one organic magnesium salt is present in a 40-60 wt % concentration and the thickener is present in a 0.2-8 wt % concentration.

17. The composition according to claim 12, wherein the at least one organic magnesium salt is present in a 40-60 wt % concentration and the thickener is present in a 0.2-8 wt % concentration.

18. The composition according to claim 13, wherein the at least one organic magnesium salt is present in a 40-60 wt % concentration and the thickener is present in a 0.2-8 wt % concentration.
